(19) United States
(12) Patent Application Publication
(10) Pub. No.: US 2021/0000731 A1
(43) Pub. Date: Jan. 7, 2021

(54) TOPICAL COMPOSITION

(71) Applicant: DSM IP ASSETS B.V., Heerlen (NL)

(72) Inventors: Guillaume Bernard LAURENT, Kaiseraugst (CH); Pascal Christian SIEBER, Kaiseraugst (CH)

(21) Appl. No.: 16/976,566

(22) PCT Filed: Feb. 28, 2019
(86) PCT No.: PCT/EP2019/054923

§ 371 Date: Aug. 28, 2020

(30) Foreign Application Priority Data
Mar. 5, 2018 (EP) 18159969.7

(51) Int. Cl. A61K 8/81; A61K 8/60; A61Q 19/00; A61K 8/06; A61K 8/04

(52) U.S. Cl. CPC A61K 8/8158; A61K 8/60; A61Q 19/00; A61K 8/064; A61Q 5/02; A61K 8/042; A61K 2800/594; A61K 2800/42; A61K 8/062

(57) ABSTRACT

The present invention relates to topical compositions comprising a saccharide isomerate and at least one acryloyldimethyltaurate polymer comprising alkali metal ions and/or ammonium ions, characterized in that the ratio of alkali metal ions to ammonium ions in the acryloyldimethyltaurate moieties in the at least one polymer is at least 1 as well as to the use of an alkali metal acryloyldimethyltaurate polymer to suppress discoloration in topical compositions comprising a saccharide isomerate and an ammonium acryloyldimethyltaurate polymer.

The present invention relates to topical compositions comprising a saccharide isomerate and at least one acryloyldimethyltaurate polymer comprising alkali metal and/or ammonium ions, characterized in that the ratio of alkali metal cations to ammonium ions in the acryloyldimethyltaurate moieties in the at least one polymer is at least 1 as well as to the use of an alkali metal acryloyldimethyltaurate polymer to suppress discoloration in topical compositions comprising a saccharide isomerate and an ammonium acryloyldimethyltaurate polymer.

Saccharide isomerate (INCI) is used topical compositions to exert beneficial physiological effects, such as short and long-lasting moisturization.

Ammonium acryloyldimethyltaurate polymers are synthetic polymers broadly used in topical compositions used for cosmetic applications.

However, in topical compositions comprising an ammonium acryloyldimethyltaurate polymer in combination with saccharide isomerate an unwanted discoloration has been observed upon storage which is not appreciated by the end consumer.

In accordance with the present invention it has now been found that the discoloration of topical compositions containing a saccharide isomerate and an ammonium acryloyldimethyltaurate polymer can effectively be reduced by the addition of an alkali metal acryloyldimethyltaurate polymer without however significantly altering other properties of the composition such as the viscosity and/or texture.

Thus, in a first embodiment the present invention relates to topical composition comprising a saccharide isomerate and at least one acryloyldimethyltaurate polymer comprising alkali metal and/or ammonium ions, characterized in that the ratio (mole) of alkali metal ions to ammonium ions in the acryloyldimethyltaurate moieties in the at least one polymer is at least 1.

In another embodiment, the present invention relates to the use of at least one acryloyldimethyltaurate polymer comprising alkali metal and/or ammonium ions wherein the ratio of alkali metal cations to ammonium ions in the acryloyldimethyltaurate moieties in the at least one polymer is at least 1 to suppress discoloration in topical compositions comprising a saccharide isomerate.

In a further embodiment, the present invention relates to the use of at least one alkali metal acryloyldimethyltaurate polymer to suppress discoloration in topical compositions, whereby the topical composition comprises a saccharide isomerate and an ammonium acryloyldimethyltaurate polymer.

In a further embodiment, the present invention relates to a method of suppressing the discoloration in a topical composition comprising a saccharide isomerate and an ammonium acryloyldimethyltaurate polymer, said method comprising the addition of an alkali metal acryloyldimethyltaurate polymer to said composition and optionally appreciating the effect.

The term ‘topical’ as used herein is understood here to mean external application to keratinous substances, which are in particular the skin, scalp, eyelashes, eyebrows, nails, mucous membranes and hair, preferably the skin of a human.

As the topical compositions according to the invention are intended for topical application, it is well understood that they comprise a physiologically acceptable medium, i.e. a medium compatible with keratinous substances, such as the skin, mucous membranes, and keratinous fibers. In particular, the physiologically acceptable medium is a cosmetically acceptable carrier.

The term ‘cosmetically acceptable carrier’ refers to all carriers and/or excipients and/or diluents conventionally used in topical cosmetic compositions such as in particular in skin care preparations.

Saccharide isomerate (CAS 100843-69-4) is a well-known cosmetic agent with unique binding mechanism to the skin used for short and long-lasting moisturization. Saccharide isomerate is e.g. commercially available under the trademark PENTAVITIN® from DSM Nutritional Products Ltd or Hyanify from Lipotec. The most preferred saccharide isomerate in all embodiments of the present invention is PENTAVITIN® which comprises as active ingredient a saccharide isomerate consisting essentially of glucose, fructose mannose and galactose.

The term ‘acryloyldimethyltaurate polymers’ as used herein refers to all cosmetically acceptable homopolymers, copolymers and crosslinked polymers, the monomers of which are at least partially composed of acryloyl dimethyltauric acid monomers respectively acryloyldimethyltaurate monomers, the latter being preferred. Such acryloyldimethyltaurate polymers are well known to a person skilled in the art and typically added to topical compositions as gelling agent and/or thickener.

Formula (I) depicts said preferred acryloyldimethyltaurate monomers:

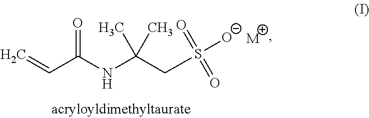

with M being an alkali metal ion or an ammonium ion, preferably a sodium cation or an ammonium ion.

The alkali metal ions and/or the ammonium ions can be introduced into the acryloyl dimethyltaurate polymers by monomers of formula (I) or by neutralisation of a polymers obtainable by using acryloyl dimethyltauric acid monomers with a base such as preferably ammonia or sodium hydroxide.

The number average molecular weight Mn (g/mol) of the acryloyl dimethyltaurate polymers according to the present invention is preferably >1000 or >10′000 such as in the range of 1000 to 1′00000 or in the range of 10′000 to 500′000.

The term ‘supress/suppressing discoloration’ refers to a reduced discoloration of the topical composition according to the present invention compared to control. The degree of discoloration can be illustrated via the Δb-value as illustrated in the example.

The amount of the saccharide isomerate in the topical compositions according to the present invention is preferably in the range from 0.01 to 10 wt.-%, more preferably in the range from 0.1 to 7.5 wt.-%, most preferably in the range from 0.2 to 5 wt.-%, based on the total weight of the topical composition.

The amount (total) of the at least one acryloyldimethyltaurate polymer in the topical compositions according to the present invention is preferably in the range from 0.01 to 10 wt.-%, more preferably in the range from 0.1 to 5 wt.-%, most preferably in the range from 0.2 to 3 wt.-% such as in the range from 0.5 to 2 wt.-% or in the range from 0.5 to 1 wt.-%, based on the total weight of the topical composition.

In a preferred embodiment, at least two different acryloyldimethyltaurate polymers are used in the topical compositions according to the present invention, wherein even more preferably one acryloyldimethyltaurate polymer is a sodium acryloyldimethyltaurate polymer and the other one is an ammonium acryloyldimethyltaurate polymer. In a particular advantageous embodiment, the weight-ratio (w/w) of the sodium acryloyldimethyltaurate polymer to the ammonium acryloyldimethyltaurate polymer in all embodiments of the present invention is selected in the range from 50:1 to 0.5:1, preferably in the range of 50:1 to 1:1, more preferably in the range from 25:1 to 1:1, most preferably in the range from 10:1 to 1:1 such as in the range of 5:1 to 1:1. Further suitable ranges include from 10:1 to 0.5:1 such as in the range of 5:1 to 0.5:1.

Particularly suitable acryloyldimethyltaurate polymers according to the present invention are all sodium and/or ammonium acryloyldimethyltaurate polymers conventionally used in cosmetics, e.g. Acrylamide/Sodium Acryloyldimethyltaurate Copolymer [CAS 38193-60-1], Acrylamide/Sodium Acryloyldimethyltaurate/Acrylic Acid Copolymer [CAS 1453194-38-1; 1417986-34-5], Ammonium Acryloyldimethyltaurate/Beheneth-25 Methacrylate Crosspolymer, Ammonium Acryloyldimethyltaurate/Carboxyethyl acrylate Crosspolymer, Ammonium Acryloyldimethyltaurate/Laureth-7 Methacrylate Copolymer [CAS 683748-07-4], Ammonium Acryloyldimethyltaurate/Steareth-25 Methacrylate Crosspolymer, Ammonium Acryloyldimethyltaurate/Steareth-8 Methacrylate Copolymer, Ammonium Acryloyldimethyltaurate/Vinyl formamide Copolymer [CAS 600172-76-7], Ammonium Acryloyldimethyltaurate/VP polymer [CAS 1509895-12-8; 53845-64-0], Ammonium Polyacryloyldimethyl Taurate [CAS 62152-14-1], Dimethylacrylamide/Sodium Acryloyldimethyltaurate Crosspolymer [CAS 869772-40-7; 92815-96-8], HEA/Sodium Acryloyldimethyltaurate/Steareth-20 Methacrylate Copolymer [CAS 1562414-82-7], Hydroxyethyl Acrylate/Sodium Acryloyldimethyltaurate Copolymer [CAS 111286-86-3], Polyacryloyldimethyltaurate Polyoxymethylene Melamine, Sodium Acrylate/Acryloyldimethyltaurate/Dimethylacrylamide Crosspolymer [CAS 187725-30-0], Sodium Acrylate/Sodium Acryloyldimethyl Taurate Copolymer [CAS 77019-71-7], Sodium Acrylate/Sodium Acryloyldimethyl Taurate/Acrylamide Copolymer [CAS 1453194-38-1; 1417986-34-5], Sodium Acryloyl Dimethyl Taurate/PEG-8 Diacrylate Crosspolymer, Sodium Acryloyldimethyl Taurate/Acrylamide/VP Copolymer [CAS 92815-97-9], Sodium Acryloyldimethyltaurate/Methacrylamidolauric Acid Copolymer [CAS 882176-38-7], Sodium Acryloyldimethyltaurate/VP Crosspolymer, and Sodium Polyacryloyldimethyltaurate [CAS 35641-59-9].

Such acryloyldimethyltaurate polymers are e.g. commercially available under the tradenames Multicare MP 100KC from Vignon, Aristoflex® AVC, Aristoflex® HMB, Aristoflex® AVC-1 or Aristoflex® AVS from Clariant and SEPINOV™ WEO, SEPINOV™ EMT 10 or SEPIPLUS™ S from Seppic.

Preferably, in all embodiments of the present invention the acryloyldimethyltaurate polymers are copolymers of sodium and/or ammonium acryloyldimethyltaurate with at least one other vinylic monomer. Such vinylic monomers are well known to a person in the art and encompass e.g. acrylates such as methyl acrylate, ethyl acrylate, hydroxyethyl acrylate or beheneth-25 methacrylate, acrylamides, vinylformamide and vinylpyrrolidone without being limited hereto. Particular preferred vinylic monomers according to the present invention are vinylpyrrolidone, beheneth-25 methacrylate, vinylformamide and/or hydroxyethylacrylate. Most preferred is beheneth-25 methacrylate and/or hydroxyethylacrylate.

The most preferred acryloyldimethyltaurate polymers in all embodiments of the present invention are Hydroxyethylacrylate/Sodium Acryloyldimethyltaurate Copolymer and Ammonium Acryloyldimethyltaurate/Beheneth-25 Methacrylate Crosspolymer.

If desired, a proportion of acryloyldimethyltaurate polymers according to the present invention may be replaced by other gelling agents. Preferably, however, the acryloyldimethyltaurate polymers are the sole gelling agents in the topical compositions according to the present invention.

Most preferably in all embodiments of the present invention a combination of Hydroxyethylacrylate/Sodium Acryloyldimethyltaurate Copolymer and Ammonium Acryloyldimethyltaurate/Beheneth-25 Methacrylate Crosspolymer is used.

Even more preferably the weight-ratio (w/w) between the Hydroxyethylacrylate/Sodium Acryloyldimethyltaurate Copolymer and the Ammonium Acryloyldimethyltaurate/Beheneth-25 Methacrylate Crosspolymer is selected in the range from 50:1 to 1:1, more preferably in the range from 25:1 to 1:1, most preferably in the range from 10:1 to 1:1. Further preferred ranges are 10:1 to 2:1, 5:1 to 1:1 or 5:1 to 2:1 as well as 10:1 to 0.5:1, 5:1.5 to 0.5:1 or 5:1 to 0.5:0.1.

In particular, the topical composition according to the present invention are cosmetic or pharmaceutical compositions, and preferably cosmetic (non-therapeutic) compositions.

In one embodiment, the topical compositions according to the present invention are applied to mammalian keratinous tissue such as in particular to human skin or the human scalp and hair.

The term “cosmetic composition” as used in the present application refers to cosmetic compositions as defined under the heading “Kosmetika” in Römpp Lexikon Chemie, 10th edition 1997, Georg Thieme Verlag Stuttgart, New York as well as to cosmetic compositions as disclosed in A. Domsch, “Cosmetic Compositions”, Verlag für chemische Industrie (ed. H. Ziolkowsky), 4th edition, 1992.

The topical compositions according to the present invention are preferably prepared by admixing at least one acryloyldimethyltaurate polymer, wherein the ratio (mole) of alkali metal ions to ammonium ions in the acryloyldimethyltaurate moieties in the at least one polymer is at least 1 with a cosmetically acceptable carrier. Preferably in all topical compositions according to the present invention two different acryloyldimethyltaurate polymers are used, one alkali metal acryloyldimethyltaurate polymer and one ammonium acryloyldimethyltaurate polymer, most preferably in the amounts and ratios as defined herein.

In a particular preferred embodiment, the topical compositions according to the present invention are prepared by admixing a sodium acryloyldimethyltaurate polymer and an ammonium acryloyldimethyltaurate polymer with all the definitions and preferences as given herein with a cosmetically acceptable carrier.

In one embodiment, the topical compositions according to the present invention are an O/W emulsion, a W/O emulsion, a gel, a shampoo preparation or a hair conditioner.

Descriptions of O/W emulsions, W/O emulsions, gels, shampoo preparations or hair conditioners are given below.

In a further embodiment, the invention relates to a method of treating the skin and/or the scalp, said method comprising the steps of contacting the skin and/or scalp with a topical composition according to anyone of the embodiments as given herein.

The topical compositions according to the present invention are generally prepared by admixing saccharide isomerate and the at least one acryloyldimethyltaurate polymer in the amounts indicated herein with a suitable carrier.

The exact amount of carrier will depend upon the actual level of saccharide isomerate and the at least one acryloyldimethyltaurate polymer and any other optional ingredients that one of ordinary skill in the art would classify as distinct from the carrier (e.g., other active ingredients).

In an advantageous embodiment, the cosmetic or pharmaceutical compositions according to the present invention comprise from 50% to 99%, preferably from 60% to 98%, more preferably from 70% to 98%, such as in particular from 80% to 95% of a carrier, based on the total weight of the topical composition.

In a particular advantageous embodiment, the carrier consists furthermore of at least 40 wt.-%, more preferably of at least 50 wt.-%, most preferably of at least 55 wt.-% of water, such as in particular of 55 to 90 wt.-% of water.

The compositions of the invention (including the carrier) may comprise conventional adjuvants and additives, such as preservatives/antioxidants, fatty substances/oils, organic solvents, silicones, thickeners, softeners, emulsifiers, antifoaming agents, aesthetic components such as fragrances, surfactants, fillers, anionic, cationic, nonionic or amphoteric polymers or mixtures thereof, propellants, acidifying or basifying agents, dyes, colorings/colorants, abrasives, absorbents, chelating agents and/or sequestering agents, essential oils, skin sensates, astringents, pigments or any other ingredients usually formulated into such compositions.

In accordance with the present invention, the compositions according to the invention may also comprise further cosmetically active ingredients conventionally used in cosmetic or pharmaceutical compositions. Exemplary active ingredients encompass UV-filters, agents for the prevention or reduction of inflammation; firming, moisturizing, soothing, and/or energizing agents as well as agents to improve elasticity and skin barrier.

Examples of cosmetic excipients, diluents, adjuvants, additives as well as active ingredients commonly used in the skin care industry which are suitable for use in the cosmetic compositions of the present invention are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The necessary amounts of the active ingredients as well as the excipients, diluents, adjuvants, additives etc. can, based on the desired product form and application, easily be determined by the skilled person. The additional ingredients can either be added to the oily phase, the aqueous phase or separately as deemed appropriate.

The cosmetically active ingredients useful herein can in some instances provide more than one benefit or operate via more than one mode of action.

Of course, one skilled in this art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

Preferably, the cosmetic or pharmaceutical compositions according to the invention are in the form of a suspension or dispersion in solvents or fatty substances, or alternatively in the form of an emulsion or micro emulsion (in particular of O/W- or W/O-type), PIT-emulsion, nano emulsion, multiple emulsion (e. g. O/W/O- or W/O/W-type), pickering emulsion, hydrogel, lipogel, one- or multiphase solution or vesicular dispersion.

The cosmetic or pharmaceutical compositions in accordance with the invention can be in the form of a liquid, lotion, a thickened lotion, a gel, a cream, a milk, an ointment or a paste.

The cosmetic or pharmaceutical compositions according to the invention have a pH in the range from 3 to 10, preferably in the range of pH from 3-8, most preferred in the range from 3 to 7.5. In another preferred embodiment the pH is in the range from 4 to 6. The pH is adjusted by methods known to a person skilled in the art, e.g. by using an acid such as a hydroxy acid including glycolic acid, lactic acid, malic acid, citric acid and tartaric acid or a base such as e.g. sodium or potassium hydroxide or ammonium hydroxide or amines such as triethanolamine or tromethamine as well as mixtures thereof.

Preferably, the compositions according to the present invention contain citric acid in an amount of at least 0.0001 wt.-%, such as e.g. in an amount from 0.01 to 1 wt.-%, in particular in an amount from 0.01 to 0.5 wt.-%, which is used for pH adjustment.

The topical compositions according to the present invention advantageously comprise at least one preservatives or preservative booster. Preferably, the preservatives respectively the preservative boosters are selected from the group of phenoxyethanol, ethylhexylglycerin, hydroxyacetophenone, glyceryl caprylate, caprylyl glycol, 1,2-hexanediol, propanediol, propylene glycol as well as mixtures thereof. When present, the preservative respectively preservative booster is preferably used in an amount from 0.01 to 2 wt.-%, more preferably in an amount from 0.05 to 1.5 wt.-%, most preferably in an amount from 0.1 to 1.0 wt.-%, based on the total weight of the topical composition. It is particularly preferred, that the topical compositions according to the present invention do not contain any further/other preservatives such as e.g. parabens and/or methylisothiazolidine.

The topical compositions according to the present invention are in particular skin care preparations, functional preparations and/or hair care preparations. Particular preferred topical compositions in all embodiments of the present invention are skin care preparations.

Examples of skin care preparations are, in particular, light protective preparations (sun care products), anti-ageing preparations, preparations for the treatment of photo-ageing, body oils, body lotions, body gels, treatment creams, skin protection ointments, moisturizing preparations such as moisturizing gels or moisturizing sprays, face and/or body moisturizers, as well as skin lightening preparations.

Examples of functional preparations are cosmetic compositions containing active ingredients such as hormone preparations, vitamin preparations, vegetable extract preparations, anti-ageing preparations, sunscreen preparations and/or antimicrobial (antibacterial or antifungal) preparations without being limited thereto.

Preferred skin care preparations in all are moisturizing sprays as well as face and/or body moisturizers.

In one embodiment, the topical compositions according to the present invention are O/W emulsions, W/O emulsions and/or gels such as shower gels or hair gels.

Particular advantageous topical compositions according to the present invention are cream gels comprising at least 60 wt.-%, more preferably at least 70 wt.-%, most preferably at least 80 wt.-% of water, based on the total weight of the topical composition. Such compositions furthermore comprise preferably less than 30 wt.-%, more preferably less than 20 wt.-%, most preferably less than 10 wt.-% of cosmetic oil(s), based on the total weight of the topical composition. Such cream gels preferably have a pH in the range from 3 to 8, preferably in the range of 4 to 6 and most preferably in the range of 5 to 6.

Examples of hair care preparations which are suitable according to the invention are shampoos, hair conditioners (also referred to as hair rinses), hairdressing compositions, hair tonics, hair regenerating compositions, hair lotions, water wave lotions, hair sprays, hair creams, hair gels, hair oils, hair pomades or hair brilliantines. Accordingly, these are always preparations which are applied to the hair and the scalp for a shorter or longer time depending on the actual purpose for which they are used.

If the hair care preparations according to the invention are supplied as shampoos, these can be clear liquids, opaque liquids (with pearly luster effect), in cream form, gel-like or else in powder form or in tablet form, and as aerosols. The surfactant raw materials on which these shampoos are based can be anionic, nonionic and amphoteric in nature and also be present in combinations of these substances.

Examples of anionic surfactants suitable for the incorporation into the shampoo preparations according to the present invention are C₁₀₋₂₀ alkyl- and alkylenecarboxylates, alkyl ether carboxylates, fatty alcohol sulfates, fatty alcohol ether sulfates, alkylolamide sulfates and sulfonates, fatty acid alkylolamide polyglycol ether sulfates, alkanesulfonates and hydroxyalkanesulfonates, olefinsulfonates, acyl esters of isothionates, alpha-sulfo fatty acid esters, alkylbenzenesulfonates, alkylphenol glycol ether sulfonates, sulfosuccinates, sulfosuccinic monoesters and diesters, fatty alcohol ether phosphates, protein-fatty acid condensation products, alkyl monoglyceride sulfates and sulfonates, alkyl glyceride ether sulfonates, fatty acid methyltaurides, fatty acid sarcosinates, and sulforicinoleates. These compounds and their mixtures are used in the form of their salts which are soluble in water or dispersible in water, for example the sodium, potassium, magnesium, ammonium, mono-, di- and triethanolammonium salts.

Examples of suitable nonionic surfactants which can be used as detergent substances are fatty alcohol ethoxylates (alkylpolyethylene glycols); alkylphenol polyethylene glycols; alkyl mercaptan polyethylene glycols; fattyamine ethoxylates (alkylaminopolyethylene glycols); fatty acid ethoxylates (acylpolyethylene glycols); polypropylene glycol ethoxylates (Pluronic); fatty acid alkylolamides (fatty acid amide polyethylene glycols); sucrose esters; sorbitol esters and polyglycol ether.

Examples of amphoteric surfactants which can be added to the shampoos are N—(C₁₂-C₁₈-alkyl)-.beta.-aminopropionates and N—(C₁₂-C₁₈-alkyl)-.beta.-iminodipropionates as alkali metal and mono-, di- and trialkylammonium salts; N-acylamidoalkyl-N,N-dimethylacetobetaine, preferably N—(C₈-C₁₈-acyl)amidopropyl-N, N-dimethylacetobetaine; C₁₂-C₁₈-alkyldimethylsulfopropylbetaine; amphoteric surfactants based on imidazoline (commercial name: Miranol®, Steinapon®), preferably the sodium salt of 1-(β-carboxymethyloxyethyl)-1-(carboxymethyl)-2-laurylimidazolinium; amine oxide, for example C₁₂-C₁₈-alkyldimethylamine oxide, fatty acid amidoalkyldimethylamine oxide.

The hair care preparations according to the invention can additionally contain further additives customary in hair care such as for example perfumes, colorants, also those which simultaneously dye or tint the hair, solvents, opacifying agents and pearly luster agents, for example esters of fatty acids with polyols, magnesium and zinc salts of fatty acids, dispersions based on copolymers, thickening agents such as sodium, potassium and ammonium chloride, sodium sulfate, fatty acid alkylolamides, cellulose derivatives, natural rubbers, also plant extracts, protein derivatives such as gelatin, collagen hydrolysates, polypeptides with a natural or synthetic basis, egg yolk, lecithin, lanolin and lanolin derivatives, fats, oils, fatty alcohols, silicones, deodorizing agents, substances with antimicrobial activity, substances with antiseborrhoeic activity, substances with keratolytic and keratoplastic effect, such as, for example, sulfur, salicylic acid and enzymes as well as further anti-dandruff agents such as olamine, climbazol, zink pyrithion, ketoconazole, salicylic acid, sulfur, tar preparations, derivatives of undecenic acid, extracts of nettel, rosmary, cottonwood, birch, walnut, willow bark and/or arnica.

The shampoos are produced in a manner known per se by mixing the individual components and where necessary further processing appropriate for the particular type of preparation.

Examples of hair care preparations according to the invention and which may be mentioned are hair conditioners, hair tonics and hair regenerating compositions, which are rinsed off from the hair after a certain time or, depending on the formulation, can also remain on the hair.

The following examples are provided to further illustrate the compositions and effects of the present invention. These examples are illustrative only and are not intended to limit the scope of the invention in any way.

### EXAMPLE

All trials have been performed in the cream-gel base formula shown in table 1 which has been prepared according to standard methods in the art. The pH of all formulations was adjusted to pH 5.5. Afterwards all samples were stored at 40° C. and 50° C. for 3 months. Then the colour stability was assessed by calculating the Δb* values by measuring the L*a*b* values (CIELAB system) at t0 and after 3 months storage, which is an indicator of the discoloration of the sample.

The viscosity has been determined by Brookfield viscometer, at 25° C., 10 rpm.

TABLE 1

| Phase | Ingredient trade Name | INCI Name | % |
|---|---|---|---|
| A | WATER DEM | AQUA | Add 100 |
| A | Glycerin 1,23 (86.5%) Ph. Eur | GLYCERIN | 3.00 |
| A | Euxyl PE 9010 | PHENOXYETHANOL; ETHYLHEXYLGLYCERIN | 1.00 |
| A | PENTAVITIN ® | SACCHARIDE ISOMERATE; AQUA; CITRIC | 3.00 |
| | | ACID; SODIUM CITRATE | |
| B | Myritol 318 | CAPRYLIC/CAPRIC TRIGLYCERIDE | 5.00 |
| | Polymers (see Table 2) | | X % (see table 2) |
| C | Natriumhydroxid 10% Solution | SODIUM HYDROXIDE; AQUA | Up to pH 5.5 |
| C | Citric Acid 25% solution | CITRIC ACID; AQUA | Up to pH 5.5− |

TABLE 2
Concentration of polymers used in the formula (table 1) and results

| | | Trial 1 | Trial 2 | Trial 4 | Trial 5 | Trial 6 | Trial 7 |
|---|---|---|---|---|---|---|---|
| Polymers | INCI | % | | | | | |
| Aristoflex AVC | AMMONIUM ACRYLOYLDIMETHYL- | 1 | | 0.5 | | 0.3 | |
| | TAURATE/VP COPOLYMER | | | | | | |
| Aristoflex HMB | AMMONIUM | | 1 | | 0.5 | | 0.3 |
| | ACRYLOYLDIMETHYLTAURATE/ | | | | | | |
| | BEHENETH-25 METHACRYLATE | | | | | | |
| | CROSSPOLYMER | | | | | | |
| Sepinov EMT 10 | HYDROXYETHYL ACRYLATE/ | | | 0.5 | 0.5 | 0.7 | 0.7 |
| | SODIUM ACRYLOYLDIMETHYL- | | | | | | |
| | TAURATE COPOLYMER | | | | | | |
| Viscosity 25° C. | | 29400 | 23100 | 21720 | 16160 | 17000 | 12800 |
| (mPa · s) | | | | | | | |
| Δb* 40° C. | | 2.49 | 1.27 | 1.22 | 1.17 | 0.97 | 0.89 |
| Δb* 50° C. | | 5.47 | 3.02 | 2.85 | 2.76 | 2.55 | 2.33 |

As can be retrieved from the data outlined in table 2, the partly replacement of an ammonium acryloyldimethyltaurate polymer by a sodium acryloyldimethyltaurate polymer results in a significantly reduced discoloration of the respective composition.

1. A topical composition comprising a saccharide isomerate and at least one acryloyldimethyltaurate polymer comprising alkali metal ions and/or ammonium ions, characterized in that the ratio of alkali metal cations to ammonium ions in the acryloyldimethyltaurate moieties in the at least one polymer is at least 1.

2. The topical composition according to claim 1, wherein the amount of the saccharide isomerate is selected in the range from 0.01 to 10 wt.-%, preferably in the range from 0.1 to 7.5 wt.-%, most preferably in the range from 0.2 to 5 wt.-%, based on the total weight of the topical composition.

3. The topical composition according to claim 1, wherein the amount of the at least one acryloyldimethyltaurate polymer is selected in the range from 0.01 to 10 wt.-%, preferably in the range from 0.1 to 5 wt.-%, most preferably in the range from 0.2 to 3 wt.-%, based on the total weight of the topical composition.

4. The topical composition according to claim 1, wherein the alkali metal ion is a sodium cation.

5. The topical composition according to claim 1, wherein the at least one acryloyldimethyltaurate polymer is a mixture of a sodium acryloyldimethyltaurate polymer and an ammonium acryloyldimethyltaurate polymer.

6. The topical composition according to claim 5, wherein the ratio (w/w) of the sodium acryloyldimethyltaurate polymer to the ammonium acryloyldimethyltaurate polymer is in the range from 50:1 to 1:1, preferably in the range from 25:1 to 1:1, most preferably in the range from 10:1 to 1:1.

7. The topical composition according to claim 1, wherein the acryloyldimethyltaurate polymer is selected from the group of copolymers of sodium and/or ammonium acryloyldimethyltaurate with a vinylic monomer.

8. The topical composition according to claim 7, wherein the vinylic monomer is selected from the group consisting of vinylpyrrolidone, beheneth-25 methacrylate, vinylformamide and hydroxyethylacrylate as well as mixtures thereof.

9. The topical composition according to claim 1, wherein the at least one acryloyldimethyltaurate polymer is a mixture of Hydroxyethylacrylate/Sodium Acryloyldimethyltaurate Copolymer and Ammonium Acryloyldimethyltaurate/Beheneth-25 Methacrylate Crosspolymer.

10. The topical composition according to claim 1, wherein the composition is an O/W emulsion, a W/O emulsion, a gel, a shampoo preparation or a hair conditioner.

11. The topical composition according to claim 10, wherein the composition is gel, preferably a cream gel comprising at least 60 wt.-%, more preferably at least 70 wt.-%, most preferably at least 80 wt.-% of water, based on the total weight of the topical composition.

12. A method of treating the skin and/or the scalp of a human, said method comprising the steps of contacting the skin and/or scalp with a topical composition according to claim 1.

13. Use of at least one sodium acryloyldimethyltaurate polymer to suppress discoloration in topical compositions, whereby the topical composition comprises a saccharide isomerate and an ammonium acryloyldimethyltaurate polymer.

14. A method of suppressing the discoloration in a topical composition comprising a saccharide isomerate and an ammonium acryloyldimethyltaurate polymer, said method comprising the addition of a sodium acryloyldimethyltaurate polymer to said topical composition
